(19) United States
(12) Patent Application Publication
(10) Pub. No.: US 2009/0099019 A1
(43) Pub. Date: Apr. 16, 2009

(54) Thiazolecarboxanilides

(76) Inventors: Jochen Dietz, Mannheim (DE); Markus Gewehr, Kastellaun (DE); Thomas Grote, Wachenheim (DE); Wassilios Grammenos, Ludwigshafen (DE); Udo Hunger, Mannheim (DE); Bernd Muller, Frankenthal (DE); Frank Schieweck, Hessheim (DE); Anja Schwogler, Mannheim (DE); Jan Klaas Lohmann, Mannheim (DE); Joachim Rheinheimer, Ludwigshafen (DE); Jens Renner, Mannheim (DE); Peter Schafer, Ottersheim (DE)

Correspondence Address: BIRCH STEWART KOLASCH & BIRCH, PO BOX 747, FALLS CHURCH, VA, 22040-0747, US

(21) Appl. No.: 11/920,417

(22) PCT Filed: May 16, 2006
(86) PCT No.: PCT/EP2006/062345

§ 371 Date: Nov. 15, 2007

(30) Foreign Application Priority Data
May 18, 2005 (DE) 10 2005 023 606.5

(51) Int. Cl. A01N 43/78; C07D 277/56

(52) U.S. Cl. 504/100; 548/200; 514/365

(57) ABSTRACT

Thiazolecarboxanilides of the formula I

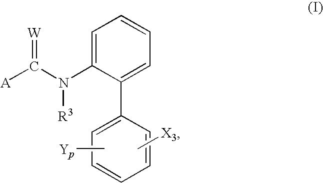

in which the variables are as defined below:
A is

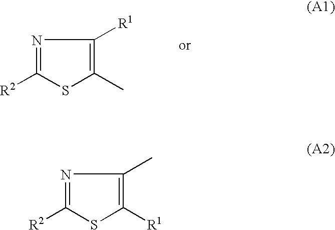

X is halogen;
Y is cyano, nitro, C₁-C₄-alkyl, C₁-C₄-haloalkyl, methoxy or methylthio;
p is 0, 1;
R¹ is hydrogen, halogen, C₁-C₄-alkyl, C₁-C₄-haloalkyl;
R² is hydrogen, methyl, halogen;
R³ is hydrogen, methyl, ethyl;
W is O, S;
and processes for preparing these compounds, compositions and seed comprising them and methods for controlling harmful fungi.

The present invention relates to thiazolecarboxanilides of the formula I

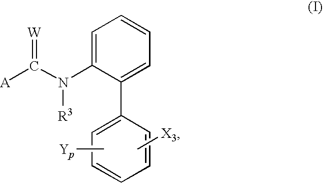

in which the variables are as defined below:
A is

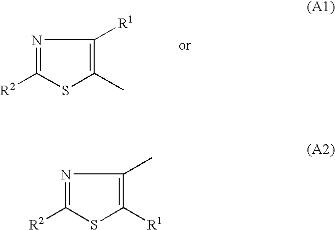

X is halogen;

Y is cyano, nitro, C₁-C₄-alkyl, C₁-C₄-haloalkyl, methoxy or methylthio;

p is 0 or 1;

R¹ is hydrogen, halogen, C₁-C₄-alkyl or C₁-C₄-haloalkyl;

R² is hydrogen, methyl or halogen;

R³ is hydrogen, methyl or ethyl;

W is oxygen or sulfur.

Here, the substituents X may independently of one another have different meanings.

Moreover, the invention relates to processes for preparing these compounds, to compositions comprising them and to methods for their use for controlling harmful fungi.

Thiazolecarboxanilides having fungicidal action are known from the literature. Thus, for example, EP-A 545 099 and EP-A 589 301 describe biphenylanilides of this type which are monosubstituted at the biphenyl group.

WO 03/066609 describes specific trifluoromethylthiazolylcarboxanilides and their fungicidal action. The compounds described are disubstituted at the biphenyl group.

WO 03/066610 describes specific difluoromethylthiazolylcarboxanilides which are mono- or disubstituted at the biphenyl group.

It was an object of the present invention to provide thiazolecarboxanilides whose fungicidal action is better than that of the compounds of the prior art.

We have found that this object is achieved by the compounds I defined at the outset.

Moreover, we have found processes for preparing these compounds, compositions comprising them and methods for their use for controlling harmful fungi.

The compounds of the formula I can be present in various crystal modifications which can differ in biological activity. They are likewise subject-matter of the present invention.

The compounds I are generally obtained by reacting a carbonyl halide of the formula II in a manner known per se (for example J. March, Advanced Organic Chemistry, 2nd Ed., 382 f, McGraw-Hill, 1977) in the presence of a base with an aniline of the formula III.

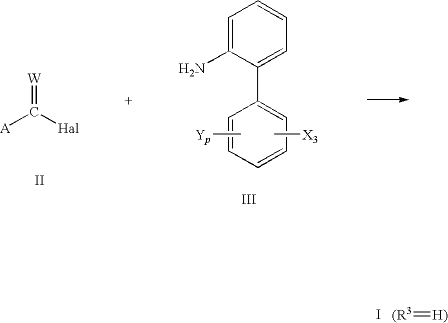

In the formula II, the radical Hal denotes a halogen atom, such as fluorine, chlorine, bromine and iodine, in particular fluorine or chlorine. This reaction is usually carried out at temperatures of from −20° C. to 100° C., preferably from 0° C. to 50° C.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chloro-benzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also methylene chloride, dimethyl sulfoxide and dimethylformamide, particularly preferably toluene, methylene chloride and tetrahydrofuran.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate and calcium carbonate, and also alkali metal bicarbonates, such as sodium bicarbonate, and organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, diisopropylethylamine and N-methyl-piperidine, pyridine, substituted pyridines, such as collidine, lutidine and 4-dimethyl-aminopyridine, and also bicyclic amines.

Particular preference is given to using triethylamine and pyridine.

The bases are generally employed in equimolar amounts, based on the compound II. However, they can also be used in an excess of from 5 mol % to 30 mol %, preferably from 5 mol % to 10 mol %, or—if tertiary amines are used—, if appropriate, as solvents.

The starting materials are generally reacted with one another in approximately equimolar amounts. In terms of yield, it may be advantageous to employ II in an excess of from 1 mol % to 20 mol %, preferably from 1 mol % to 10 mol %, based on III.

The starting materials of the formulae II and III required for preparing the compounds I are known or can be synthesized analogously to the known compounds (Helv. Chim. Acta, 60, 978 (1977); Zh. Org. Khim., 26, 1527 (1990); Heterocycles 26, 1885 (1987); Izv. Akad. Nauk. SSSR Ser. Khim., 2160 (1982); THL 28, 593 (1987); THL 29, 5463 (1988)).

Furthermore, it has been found that compounds of the formula I are obtained by reacting, in a known manner, carboxylic acids of the formula IV with an aniline of the formula III in the presence of dehydrating agents and, if appropriate, an organic base.

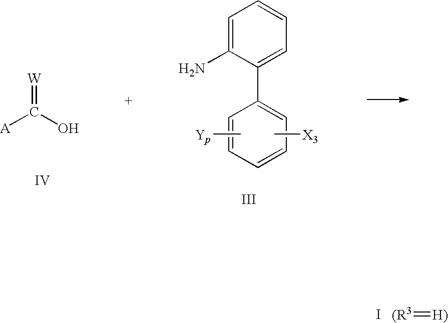

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chloro-benzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, and also dimethyl sulfoxide and dimethylformamide, particularly preferably methylene chloride, toluene and tetrahydrofuran.

It is also possible to use mixtures of the solvents mentioned.

Examples of suitable dehydrating agents are 1,1′-carbonyldiimidazole, bis(2-oxo-3-oxazolidinyl)phosphoryl chloride, carbodiimides, such as N,N′-dicyclohexylcarbodiimide, N-(3-dimethylaminopropyl)-N′-ethylcarbodiimide, phosphonium salts, such as (benzotriazol-1-yloxy)tris(dimethylamino)phosphonium hexafluorophosphate, bromotripyrrolidinophosphonium hexafluorophosphate, bromotris(dimethylamino)phosphonium hexafluorophosphate, chlorotripyrrolidinophosphonium hexafluorophosphate, uronium and thiuronium salts, such as O-(benzotriazol-1-yl)-N,N,N′,N′-tetramethyluronium hexafluorophosphate, O-(7-azabenzotriazol-1-yl)-N,N,N′,N′-tetramethyluronium hexafluorophosphate, S-(1-oxido-2-pyridyl)-N,N,N′,N′-tetramethylthiuronium tetrafluoroborate, O-(2-oxo-1(2H)pyridyl)-N,N,N′,N′-tetramethyluronium tetrafluoroborate, O-[(ethoxycarbonyl)cyanomethylenamino]-N,N,N′,N′-tetramethyluronium tetrafluoroborate, carbenium salts, such as (benzotriazol-1-yloxy)dipyrrolidinocarbenium hexafluorophosphate, (benzotriazol-1-yloxy)dipiperidinocarbenium hexafluorophosphate, O-(3,4-dihydro-4-oxo-1,2,3-benzotriazin-3-yl)-N,N,N′,N′-tetramethyluronium tetrafluoroborate, chloro-N′,N′-bis(tetramethylene)formamidinium tetrafluoroborate, chlorodipyrrolidinocarbenium hexafluorophosphate, chloro-N,N,N′,N′-bis(pentamethylene)formamidinium tetrafluoroborate, imidazolium salts, such as 2-chloro-1,3-dimethylimidazolidinium tetrafluoroborate, preferably 1,1′-carbonyldiimidazole, bis(2-oxo-3-oxazolidinyl)phosphoryl chloride, N,N′-dicyclo-hexylcarbodiimide and N-(3-dimethylaminopropyl)-N′-ethylcarbodiimide.

Examples of suitable organic bases are tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to using triethylamine and pyridine. The bases are generally employed in an excess of from 10 mol % to 200 mol %, preferably from 50 mol % to 150 mol %, based on the compound IV.

The starting materials are generally reacted with one another in approximately equimolar amounts. In terms of yield, it may be advantageous to use an excess of from 1 mol % to 20 mol %, preferably from 1 mol % to 10 mol %, of one of the compounds. The dehydrating agents are generally employed in an excess of from 5 mol % to 100 mol %, preferably from 5 mol % to 60 mol %.

The starting materials of the formulae III and IV required for preparing the compounds I are known or can be synthesized analogously to the known compounds.

The compounds I where R³═CH₃ or C₂H₅ are preferably obtained by reacting compounds of the formula I where R³═H in a known manner in the presence of a base with an alkylating agent.

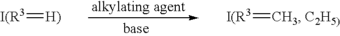

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chloro-benzene, ethers, such as diethyl ether, diisopropyl ether, tert-butyl methyl ether, dioxane, anisole and tetrahydrofuran, and also dimethyl sulfoxide and dimethylformamide, particularly preferably diethyl ether, tert-butyl methyl ether, tetrahydrofuran and dimethylformamide.

It is also possible to use mixtures of the solvents mentioned.

Examples of suitable alkylating agents are alkyl halides, such as methyl iodide, ethyl iodide, methyl bromide, ethyl bromide, methyl chloride and ethyl chloride, alkyl perfluoroalkylsulfonates, such as methyl trifluoromethylsulfonate and ethyl trifluoro-methylsulfonate, alkyl alkylsulfonates, such as methyl methylsulfonate and ethyl methylsulfonate, alkyl arylsulfonates, such as methyl p-tolylsulfonate and ethyl p-tolylsulfonate, oxonium salts, such as trimethyloxonium tetrafluoroborate and triethyloxonium tetrafluoroborate.

Particular preference is given to methyl iodide, ethyl iodide, methyl bromide, ethyl bromide, methyl chloride and ethyl chloride.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, sodium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide and potassium tert-butoxide.

Particular preference is given to using sodium carbonate, potassium carbonate, sodium hydride, potassium hydride, butyllithium and potassium tert-butoxide.

The bases are generally employed in approximately equimolar amounts, based on the compound I. However, they can also be used in an excess of from 5 mol % to 30 mol %, preferably from 5 mol % to 10 mol %.

The starting materials are generally reacted with one another in approximately equimolar amounts. In terms of yield, it may be advantageous to employ the alkylating agent in an excess of from 1 mol % to 20 mol %, preferably from 1 mol % to 10 mol %, based on I.

The compounds I in which X is sulfur can be prepared, for example, by sulfurization of the corresponding compounds I in which X is oxygen (cf. e.g. D. Petrova & K. Jakobcic, Croat. Chem. Acta 48, 49 (1976) and WO 01/42223).

With a view to their use in fungicidal compositions, suitable compounds of the formula I are those in which the substituents are as defined below:

halogen, such as fluorine, chlorine, bromine and iodine;
C₁-C₄-alkyl, such as methyl, ethyl, n-propyl, 1-methylethyl, n-butyl, 1-methylpropyl, 2-methylpropyl and 1,1-dimethylethyl;
C₁-C₄-haloalkyl is a partially or completely halogenated C₁-C₄-alkyl radical, the halogen atom(s) being in particular fluorine, chlorine and/or bromine, that is to say for example, such as chloromethyl, bromomethyl, dichloromethyl, trichloromethyl, fluoromethyl, difluoromethyl, trifluoromethyl, chlorofluoromethyl, dichlorofluoromethyl, chloro-difluoromethyl, 1-chloroethyl, 1-bromoethyl, 1-fluoroethyl, 2-fluoroethyl, 2,2-difluoro-ethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, heptafluoropropyl or nonafluoro-butyl, in particular halomethyl, particularly preferably CH₂—Cl, CH(Cl)₂, CH₂F, CHF₂, CF₃, CHFCl, CF₂Cl or CF(Cl)₂.

With a view to the biological action, particularly preferred compounds I are those in which the variables denote the following radicals:
X is F, Cl, preferably fluorine;
Y is C₁-C₄-alkyl, C₁-C₄-haloalkyl, methoxy, preferably methyl, difluoromethyl, trifluoromethyl, methoxy; very particularly preferably methyl, trifluoromethyl;
p is 0, 1, preferably 0;
R¹ is hydrogen, halogen, C₁-C₄-alkyl, C₁-C₄-haloalkyl; preferably hydrogen, F, Cl, methyl, fluoromethyl, difluoromethyl, chlorofluoromethyl, chlorodifluoromethyl, dichlorofluoromethyl, trifluoromethyl; very particularly preferably hydrogen, methyl, fluoromethyl, difluoromethyl, chlorofluoromethyl, trifluoromethyl; in particular difluoromethyl or trifluoromethyl;
R² is hydrogen, halogen, methyl; preferably hydrogen, F, Cl, methyl; very particularly preferably hydrogen, Cl or methyl;
R³ is hydrogen, methyl; preferably hydrogen;
W is oxygen.

Particular preference is given to compounds I having the following combinations of substituents, where the variables are as defined below:
X is F or chlorine;
Y is methyl, difluoromethyl, trifluoromethyl or methoxy;
p is 0, 1;
R¹ is hydrogen, F, Cl, methyl, fluoromethyl, difluoromethyl, chlorofluoromethyl, chlorodifluoromethyl, dichlorofluoromethyl, trifluoromethyl;
R² is hydrogen, F, Cl, methyl;
R³ is hydrogen or methyl;
W is oxygen.

Preference is furthermore also given to the following combinations of variables having the following meanings:
X is F or chlorine;
p is zero;
R¹ is hydrogen, F, Cl, methyl, fluoromethyl, difluoromethyl, chlorofluoromethyl, chlorodifluoromethyl, dichlorofluoromethyl or trifluoromethyl;
R² is hydrogen, F, Cl or methyl; preferably hydrogen, Cl or methyl;
R³ is hydrogen;
W is oxygen.

In particular with a view to their use as fungicides, preference is given to the compounds of the general formula I-A.

TABLE A
(I-A)

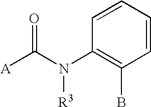

| No. | B | R¹ |
|---|---|---|
| 1 | 2-chloro-3,4-difluorophenyl | CF₃ |
| 2 | 2-chloro-4,5-difluorophenyl | CF₃ |
| 3 | 2-chloro-5,6-difluorophenyl | CF₃ |
| 4 | 2-chloro-3,5-difluorophenyl | CF₃ |
| 5 | 2-chloro-3,6-difluorophenyl | CF₃ |
| 6 | 2-chloro-4,6-difluorophenyl | CF₃ |
| 7 | 3-chloro-2,4-difiuorophenyl | CF₃ |
| 8 | 3-chloro-2,5-difluorophenyl | CF₃ |
| 9 | 3-chloro-2,6-difluorophenyl | CF₃ |
| 10 | 3-chloro-4,5-difluorophenyl | CF₃ |
| 11 | 3-chloro-4,6-difluorophenyl | CF₃ |
| 12 | 3-chloro-5,6-difluorophenyl | CF₃ |
| 13 | 4-chloro-2,3-difluorophenyl | CF₃ |
| 14 | 4-chloro-2,5-difluorophenyl | CF₃ |
| 15 | 4-chloro-2,6-difluorophenyl | CF₃ |
| 16 | 4-chloro-3,5-difluorophenyl | CF₃ |
| 17 | 2-fluoro-3,4-dichlorophenyl | CF₃ |
| 18 | 2-fluoro-4,5-dichlorophenyl | CF₃ |
| 19 | 2-ftuoro-5,6-dichlorophenyl | CF₃ |
| 20 | 2-fluoro-3,5-dichlorophenyl | CF₃ |
| 21 | 2-fluoro-3,6-dichlorophenyl | CF₃ |
| 22 | 2-fluoro-4,6-dichlorophenyl | CF₃ |
| 23 | 3-fluoro-2,4-dichlorophenyl | CF₃ |
| 24 | 3-fluoro-2,5-dichlorophenyl | CF₃ |
| 25 | 3-fluoro-2,6-dichlorophenyl | CF₃ |
| 26 | 3-fluoro-4,5-dichlorophenyl | CF₃ |
| 27 | 3-fluoro-4,6-dichlorophenyl | CF₃ |
| 28 | 3-fluoro-5,6-dichlorophenyl | CF₃ |
| 29 | 4-fluoro-2,3-dichlorophenyl | CF₃ |
| 30 | 4-fluoro-2,5-dichlorophenyl | CF₃ |
| 31 | 4-fluoro-2,6-dichlorophenyl | CF₃ |
| 32 | 4-fluoro-3,5-dichlorophenyl | CF₃ |
| 33 | 2,3,4-trichlorophenyl | CHF₂ |
| 34 | 2,3,5-trichlorophenyl | CHF₂ |
| 35 | 2,3,6-trichlorophenyl | CHF₂ |
| 36 | 2,4,5-trichlorophenyl | CHF₂ |
| 37 | 2,4,6-trichlorophenyl | CHF₂ |
| 38 | 3,4,5-trichlorophenyl | CHF₂ |
| 39 | 2,3,4-trifluorophenyl | CHF₂ |
| 40 | 2,3,5-trifluorophenyl | CHF₂ |
| 41 | 2,3,6-trifluorophenyl | CHF₂ |
| 42 | 2,4,5-trifluorophenyl | CHF₂ |
| 43 | 2,4,6-trifluorophenyl | CHF₂ |
| 44 | 3,4,5-trifluorophenyl | CHF₂ |
| 45 | 2-chloro-3,4-difluorophenyl | CHF₂ |
| 46 | 2-chloro-4,5-difluorophenyl | CHF₂ |
| 47 | 2-chloro-5,6-difluorophenyl | CHF₂ |
| 48 | 2-chloro-3,5-difluorophenyl | CHF₂ |
| 49 | 2-chloro-3,6-difluorophenyl | CHF₂ |
| 50 | 2-chloro-4,6-difluorophenyl | CHF₂ |
| 51 | 3-chloro-2,4-difluorophenyl | CHF₂ |
| 52 | 3-chloro-2,5-difluorophenyl | CHF₂ |
| 53 | 3-chloro-2,6-difluorophenyl | CHF₂ |
| 54 | 3-chloro-4,5-difluorophenyl | CHF₂ |
| 55 | 3-chloro-4,6-difluorophenyl | CHF₂ |
| 56 | 3-chloro-5,6-difluorophenyl | CHF₂ |
| 57 | 4-chloro-2,3-difluorophenyl | CHF₂ |
| 58 | 4-chloro-2,5-difluorophenyl | CHF₂ |
| 59 | 4-chloro-2,6-difluorophenyl | CHF₂ |
| 60 | 4-chloro-3,5-difluorophenyl | CHF₂ |
| 61 | 2-fluoro-3,4-dichlorophenyl | CHF₂ |
| 62 | 2-fluoro-4,5-dichlorophenyl | CHF₂ |
| 63 | 2-fluoro-5,6-dichlorophenyl | CHF₂ |
| 64 | 2-fluoro-3,5-dichlorophenyl | CHF₂ |
| 65 | 2-fluoro-3,6-dichlorophenyl | CHF₂ |
| 66 | 2-fluoro-4,6-dichlorophenyl | CHF₂ |
| 67 | 3-fluoro-2,4-dichlorophenyl | CHF₂ |
| 68 | 3-fluoro-2,5-dichlorophenyl | CHF₂ |
| 69 | 3-fiuoro-2,6-dichlorophenyl | CHF₂ |
| 70 | 3-fluoro-4,5-dichlorophenyl | CHF₂ |
| 71 | 3-fluoro-4,6-dichlorophenyl | CHF₂ |
| 72 | 3-fluoro-5,6-dichlorophenyl | CHF₂ |
| 73 | 4-fluoro-2,3-dichlorophenyl | CHF₂ |
| 74 | 4-fluoro-2,5-dichlorophenyl | CHF₂ |
| 75 | 4-fluoro-2,6-dichlorophenyl | CHF₂ |
| 76 | 4-fluoro-3,5-dichlorophenyl | CHF₂ |
| 77 | 2,3,4-trichlorophenyl | CH₂F |
| 78 | 2,3,5-trichlorophenyl | CH₂F |
| 79 | 2,3,6-trichlorophenyl | CH₂F |
| 80 | 2,4,5-trichlorophenyl | CH₂F |
| 81 | 2,4,6-trichlorophenyl | CH₂F |
| 82 | 3,4,5-trichlorophenyl | CH₂F |
| 83 | 2,3,4-trifluorophenyl | CH₂F |
| 84 | 2,3,5-trifluorophenyl | CH₂F |
| 85 | 2,3,6-trifluorophenyl | CH₂F |
| 86 | 2,4,5-trifluorophenyl | CH₂F |
| 87 | 2,4,6-trifluorophenyl | CH₂F |
| 88 | 3,4,5-trifluorophenyl | CH₂F |
| 89 | 2-chloro-3,4-difiuorophenyl | CH₂F |
| 90 | 2-chloro-4,5-difluorophenyl | CH₂F |
| 91 | 2-chloro-5,6-difluorophenyl | CH₂F |
| 92 | 2-chloro-3,5-difluorophenyl | CH₂F |
| 93 | 2-chloro-3,6-difluorophenyl | CH₂F |
| 94 | 2-chloro-4,6-difluorophenyl | CH₂F |
| 95 | 3-chloro-2,4-difluorophenyl | CH₂F |
| 96 | 3-chloro-2,5-difluorophenyl | CH₂F |
| 97 | 3-chloro-2,6-difluorophenyl | CH₂F |
| 98 | 3-chloro-4,5-difluorophenyl | CH₂F |
| 99 | 3-chloro-4,6-difluorophenyl | CH₂F |
| 100 | 3-chloro-5,6-difluorophenyl | CH₂F |
| 101 | 4-chloro-2,3-difluorophenyl | CH₂F |
| 102 | 4-chloro-2,5-difluorophenyl | CH₂F |
| 103 | 4-chloro-2,6-difluorophenyl | CH₂F |
| 104 | 4-chloro-3,5-difluorophenyl | CH₂F |
| 105 | 2-fluoro-3,4-dichlorophenyl | CH₂F |
| 106 | 2-fluoro-4,5-dichlorophenyl | CH₂F |
| 107 | 2-fluoro-5,6-dichlorophenyl | CH₂F |
| 108 | 2-fluoro-3,5-dichlorophenyl | CH₂F |
| 109 | 2-fluoro-3,6-dichlorophenyl | CH₂F |
| 110 | 2-fluoro-4,6-dichlorophenyl | CH₂F |
| 111 | 3-fluoro-2,4-dichlorophenyl | CH₂F |
| 112 | 3-fluoro-2,5-dichlorophenyl | CH₂F |
| 113 | 3-fluoro-2,6-dichlorophenyl | CH₂F |
| 114 | 3-fluoro-4,5-dichlorophenyl | CH₂F |
| 115 | 3-fluoro-4,6-dichlorophenyl | CH₂F |
| 116 | 3-fluoro-5,6-dichlorophenyl | CH₂F |
| 117 | 4-fluoro-2,3-dichlorophenyl | CH₂F |
| 118 | 4-fluoro-2,5-dichlorophenyl | CH₂F |
| 119 | 4-fluoro-2,6-dichlorophenyl | CH₂F |
| 120 | 4-fluoro-3,5-dichlorophenyl | CH₂F |
| 121 | 2,3,4-trichlorophenyl | CHFCl |
| 122 | 2,3,5-trichlorophenyl | CHFCl |
| 123 | 2,3,6-trichlorophenyl | CHFCl |
| 124 | 2,4,5-trichlorophenyl | CHFCl |
| 125 | 2,4,6-trichlorophenyl | CHFCl |
| 126 | 3,4,5-trichlorophenyl | CHFCl |
| 127 | 2,3,4-trifluorophenyl | CHFCl |
| 128 | 2,3,5-trifluorophenyl | CHFCl |
| 129 | 2,3,6-trifluorophenyl | CHFCl |
| 130 | 2,4,5-trifluorophenyl | CHFCl |
| 131 | 2,4,6-trifluorophenyl | CHFCl |
| 132 | 3,4,5-trifluorophenyl | CHFCl |
| 133 | 2-chloro-3,4-difluorophenyl | CHFCl |
| 134 | 2-chloro-4,5-difluorophenyl | CHFCl |
| 135 | 2-chloro-5,6-difluorophenyl | CHFCl |
| 136 | 2-chloro-3,5-difluorophenyl | CHFCl |
| 137 | 2-chloro-3,6-difluorophenyl | CHFCl |
| 138 | 2-chloro-4,6-difluorophenyl | CHFCl |
| 139 | 3-chloro-2,4-difluorophenyl | CHFCl |
| 140 | 3-chloro-2,5-difluorophenyl | CHFCl |
| 141 | 3-chloro-2,6-difluorophenyl | CHFCl |
| 142 | 3-chloro-4,5-difluorophenyl | CHFCl |
| 143 | 3-chloro-4,6-difluorophenyl | CHFCl |
| 144 | 3-chloro-5,6-difluorophenyl | CHFCl |
| 145 | 4-chloro-2,3-difluorophenyl | CHFCl |
| 146 | 4-chloro-2,5-difluorophenyl | CHFCl |
| 147 | 4-chloro-2,6-difluorophenyl | CHFCl |
| 148 | 4-chloro-3,5-difluorophenyl | CHFCl |
| 149 | 2-fluoro-3,4-dichlorophenyl | CHFCl |
| 150 | 2-fluoro-4,5-dichlorophenyl | CHFCl |
| 151 | 2-fluoro-5,6-dichlorophenyl | CHFCl |
| 152 | 2-fluoro-3,5-dichlorophenyl | CHFCl |
| 153 | 2-fluoro-3,6-dichlorophenyl | CHFCl |
| 154 | 2-fluoro-4,6-dichlorophenyl | CHFCl |
| 155 | 3-fluoro-2,4-dichlorophenyl | CHFCl |
| 156 | 3-fluoro-2,5-dichlorophenyl | CHFCl |
| 157 | 3-fluoro-2,6-dichlorophenyl | CHFCl |
| 158 | 3-fluoro-4,5-dichlorophenyl | CHFCl |
| 159 | 3-fluoro-4,6-dichlorophenyl | CHFCl |
| 160 | 3-fluoro-5,6-dichlorophenyl | CHFCl |
| 161 | 4-fluoro-2,3-dichlorophenyl | CHFCl |
| 162 | 4-fluoro-2,5-dichlorophenyl | CHFCl |
| 163 | 4-fluoro-2,6-dichlorophenyl | CHFCl |
| 164 | 4-fluoro-3,5-dichlorophenyl | CHFCl |
| 165 | 2,3,4-trichlorophenyl | CF₂Cl |
| 166 | 2,3,5-trichlorophenyl | CF₂Cl |
| 167 | 2,3,6-trichlorophenyl | CF₂Cl |
| 168 | 2,4,5-trichlorophenyl | CF₂Cl |
| 169 | 2,4,6-trichlorophenyl | CF₂Cl |
| 170 | 3,4,5-trichlorophenyl | CF₂Cl |
| 171 | 2,3,4-trifluorophenyl | CF₂Cl |
| 172 | 2,3,5-trifluorophenyl | CF₂Cl |
| 173 | 2,3,6-trifluorophenyl | CF₂Cl |
| 174 | 2,4,5-trifluorophenyl | CF₂Cl |
| 175 | 2,4,6-trifluorophenyl | CF₂Cl |
| 176 | 3,4,5-trifluorophenyl | CF₂Cl |
| 177 | 2-chloro-3,4-difluorophenyl | CF₂Cl |
| 178 | 2-chloro-4,5-difluorophenyl | CF₂Cl |
| 179 | 2-chloro-5,6-difluorophenyl | CF₂Cl |
| 180 | 2-chloro-3,5-difluorophenyl | CF₂Cl |
| 181 | 2-chloro-3,6-difluorophenyl | CF₂Cl |
| 182 | 2-chloro-4,6-difluorophenyl | CF₂Cl |
| 183 | 3-chloro-2,4-difluorophenyl | CF₂Cl |
| 184 | 3-chloro-2,5-difluorophenyl | CF₂Cl |
| 185 | 3-chloro-2,6-difluorophenyl | CF₂Cl |
| 186 | 3-chloro-4,5-difluorophenyl | CF₂Cl |
| 187 | 3-chloro-4,6-difluorophenyl | CF₂Cl |
| 188 | 3-chloro-5,6-difluorophenyl | CF₂Cl |
| 189 | 4-chloro-2,3-difluorophenyl | CF₂Cl |
| 190 | 4-chloro-2,5-difluorophenyl | CF₂Cl |
| 191 | 4-chloro-2,6-difluorophenyl | CF₂Cl |
| 192 | 4-chloro-3,5-difluorophenyl | CF₂Cl |
| 193 | 2-fluoro-3,4-dichlorophenyl | CF₂Cl |
| 194 | 2-fluoro-4,5-dichlorophenyl | CF₂Cl |
| 195 | 2-fluoro-5,6-dichlorophenyl | CF₂Cl |
| 196 | 2-fluoro-3,5-dichlorophenyl | CF₂Cl |
| 197 | 2-fluoro-3,6-dichlorophenyl | CF₂Cl |
| 198 | 2-fluoro-4,6-dichlorophenyl | CF₂Cl |
| 199 | 3-fluoro-2,4-dichlorophenyl | CF₂Cl |
| 200 | 3-fluoro-2,5-dichlorophenyl | CF₂Cl |
| 201 | 3-fluoro-2,6-dichlorophenyl | CF₂Cl |
| 202 | 3-fluoro-4,5-dichlorophenyl | CF₂Cl |
| 203 | 3-fluoro-4,6-dichlorophenyl | CF₂Cl |
| 204 | 3-fluoro-5,6-dichlorophenyl | CF₂Cl |
| 205 | 4-fluoro-2,3-dichlorophenyl | CF₂Cl |
| 206 | 4-fluoro-2,5-dichlorophenyl | CF₂Cl |
| 207 | 4-fluoro-2,6-dichlorophenyl | CF₂Cl |
| 208 | 4-fluoro-3,5-dichlorophenyl | CF₂Cl |
| 209 | 2,3,4-trichlorophenyl | CFCl₂ |
| 210 | 2,3,5-trichlorophenyl | CFCl₂ |
| 211 | 2,3,6-trichlorophenyl | CFCl₂ |
| 212 | 2,4,5-trichlorophenyl | CFCl₂ |
| 213 | 2,4,6-trichlorophenyl | CFCl₂ |
| 214 | 3,4,5-trichlorophenyl | CFCl₂ |
| 215 | 2,3,4-trifluorophenyl | CFCl₂ |
| 216 | 2,3,5-trifluorophenyl | CFCl₂ |
| 217 | 2,3,6-trifluorophenyl | CFCl₂ |
| 218 | 2,4,5-trifluorophenyl | CFCl₂ |
| 219 | 2,4,6-trifluorophenyl | CFCl₂ |
| 220 | 3,4,5-trifluorophenyl | CFCl₂ |
| 221 | 2-chloro-3,4-difluorophenyl | CFCl₂ |
| 222 | 2-chloro-4,5-difluorophenyl | CFCl₂ |
| 223 | 2-chloro-5,6-difluorophenyl | CFCl₂ |
| 224 | 2-chloro-3,5-difluorophenyl | CFCl₂ |
| 225 | 2-chloro-3,6-difluorophenyl | CFCl₂ |
| 226 | 2-chloro-4,6-difluorophenyl | CFCl₂ |
| 227 | 3-chloro-2,4-difluorophenyl | CFCl₂ |
| 228 | 3-chloro-2,5-difluorophenyl | CFCl₂ |
| 229 | 3-chloro-2,6-difluorophenyl | CFCl₂ |
| 230 | 3-chloro-4,5-difluorophenyl | CFCl₂ |
| 231 | 3-chloro-4,6-difluorophenyl | CFCl₂ |
| 232 | 3-chloro-5,6-difluorophenyl | CFCl₂ |
| 233 | 4-chloro-2,3-difluorophenyl | CFCl₂ |
| 234 | 4-chloro-2,5-diftuorophenyl | CFCl₂ |
| 235 | 4-chloro-2,6-difluorophenyl | CFCl₂ |
| 236 | 4-chloro-3,5-difluorophenyl | CFCl₂ |
| 237 | 2-fluoro-3,4-dichlorophenyl | CFCl₂ |
| 238 | 2-fluoro-4,5-dichlorophenyl | CFCl₂ |
| 239 | 2-fluoro-5,6-dichlorophenyl | CFCl₂ |
| 240 | 2-fluoro-3,5-dichlorophenyl | CFCl₂ |
| 241 | 2-fluoro-3,6-dichlorophenyl | CFCl₂ |
| 242 | 2-fluoro-4,6-dichlorophenyl | CFCl₂ |
| 243 | 3-fluoro-2,4-dichlorophenyl | CFCl₂ |
| 244 | 3-fluoro-2,5-dichlorophenyl | CFCl₂ |
| 245 | 3-fluoro-2,6-dichlorophenyl | CFCl₂ |
| 246 | 3-fluoro-4,5-dichlorophenyl | CFCl₂ |
| 247 | 3-fluoro-4,6-dichlorophenyl | CFCl₂ |
| 248 | 3-fluoro-5,6-dichlorophenyl | CFCl₂ |
| 249 | 4-fluoro-2,3-dichlorophenyl | CFCl₂ |
| 250 | 4-fluoro-2,5-dichlorophenyl | CFCl₂ |
| 251 | 4-fluoro-2,6-dichlorophenyl | CFCl₂ |
| 252 | 4-fluoro-3,5-dichlorophenyl | CFCl₂ |
| 253 | 2,3,4-trichlorophenyl | CH₃ |
| 254 | 2,3,5-trichlorophenyl | CH₃ |
| 255 | 2,3,6-trichlorophenyl | CH₃ |
| 256 | 2,4,5-trichlorophenyl | CH₃ |
| 257 | 2,4,6-trichlorophenyl | CH₃ |
| 258 | 3,4,5-trichlorophenyl | CH₃ |
| 259 | 2,3,4-trifluorophenyl | CH₃ |
| 260 | 2,3,5-trifluorophenyl | CH₃ |
| 261 | 2,3,6-trifluorophenyl | CH₃ |
| 262 | 2,4,5-trifluorophenyl | CH₃ |
| 263 | 2,4,6-trifluorophenyl | CH₃ |
| 264 | 3,4,5-trifluorophenyl | CH₃ |
| 265 | 2-chloro-3,4-difluorophenyl | CH₃ |
| 266 | 2-chloro-4,5-difluorophenyl | CH₃ |
| 267 | 2-chloro-5,6-difluorophenyl | CH₃ |
| 268 | 2-chloro-3,5-difluorophenyl | CH₃ |
| 269 | 2-chloro-3,6-difluorophenyl | CH₃ |
| 270 | 2-chloro-4,6-difluorophenyl | CH₃ |
| 271 | 3-chloro-2,4-difluorophenyl | CH₃ |
| 272 | 3-chloro-2,5-difluorophenyl | CH₃ |
| 273 | 3-chloro-2,6-difluorophenyl | CH₃ |
| 274 | 3-chloro-4,5-difluorophenyl | CH₃ |
| 275 | 3-chloro-4,6-difluorophenyl | CH₃ |
| 276 | 3-chloro-5,6-difluorophenyl | CH₃ |
| 277 | 4-chloro-2,3-difluorophenyl | CH₃ |
| 278 | 4-chloro-2,5-difluorophenyl | CH₃ |
| 279 | 4-chloro-2,6-difluorophenyl | CH₃ |
| 280 | 4-chloro-3,5-difluorophenyl | CH₃ |
| 281 | 2-fluoro-3,4-dichlorophenyl | CH₃ |
| 282 | 2-fluoro-4,5-dichlorophenyl | CH₃ |
| 283 | 2-fluoro-5,6-dichlorophenyl | CH₃ |
| 284 | 2-fluoro-3,5-dichlorophenyl | CH₃ |
| 285 | 2-fluoro-3,6-dichlorophenyl | CH₃ |
| 286 | 2-fluoro-4,6-dichlorophenyl | CH₃ |
| 287 | 3-fluoro-2,4-dichlorophenyl | CH₃ |
| 288 | 3-fluoro-2,5-dichlorophenyl | CH₃ |
| 289 | 3-fluoro-2,6-dichlorophenyl | CH₃ |
| 290 | 3-fluoro-4,5-dichlorophenyl | CH₃ |
| 291 | 3-fluoro-4,6-dichlorophenyl | CH₃ |
| 292 | 3-fluoro-5,6-dichlorophenyl | CH₃ |
| 293 | 4-fluoro-2,3-dichlorophenyl | CH₃ |
| 294 | 4-fluoro-2,5-dichlorophenyl | CH₃ |
| 295 | 4-fluoro-2,6-dichlorophenyl | CH₃ |
| 296 | 4-fluoro-3,5-dichlorophenyl | CH₃ |

### Table 1:

Compounds of the general formula I-A in which A is A1, R², R³ are hydrogen and R¹ and B for each individual compound correspond in each case to one row of Table A.

### Table 2:

Compounds of the general formula I-A in which A is A1, R² is methyl, R³ is hydrogen and R¹ and B for each individual compound correspond in each case to one row of Table A.

### Table 3:

Compounds of the general formula I-A in which A is A1, R² is Cl, R³ is hydrogen and R¹ and B for each individual compound correspond in each case to one row of Table A.

### Table 4:

Compounds of the general formula I-A in which A is A1, R² is F, R³ is hydrogen and R¹ and B for each individual compound correspond in each case to one row of Table A.

### Table 5:

Compounds of the general formula I-A in which A is A1, R² is hydrogen, R³ is methyl and R¹ and B for each individual compound correspond in each case to one row of Table A.

### Table 6:

Compounds of the general formula I-A in which A is A1, R², R³ are methyl and R¹ and B for each individual compound correspond in each case to one row of Table A.

### Table 7:

Compounds of the general formula I-A in which A is A1, R² is Cl, R³ is methyl and R¹ and B for each individual compound correspond in each case to one row of Table A.

### Table 8:

Compounds of the general formula I-A in which A is A1, R² is F, R³ is methyl and R¹ and B for each individual compound correspond in each case to one row of Table A.

### Table 9:

Compounds of the general formula I-A in which A is A1, R² is hydrogen, R³ is ethyl and R¹ and B for each individual compound correspond in each case to one row of Table A.

### Table 10:

Compounds of the general formula I-A in which A is A1, R² is methyl, R³ is ethyl and R¹ and B for each individual compound correspond in each case to one row of Table A.

### Table 11:

Compounds of the general formula I-A in which A is A1, R² is Cl, R³ is ethyl and R¹ and B for each individual compound correspond in each case to one row of Table A.

### Table 12:

Compounds of the general formula I-A in which A is A1, R² is F, R³ is ethyl and R¹ and B for each individual compound correspond in each case to one row of Table A.

### Table 13:

Compounds of the general formula I-A in which A is A2, R², R³ are hydrogen and R¹ and B for each individual compound correspond in each case to one row of Table A.

### Table 14:

Compounds of the general formula I-A in which A is A2, R² is methyl, R³ is hydrogen and R¹ and B for each individual compound correspond in each case to one row of Table A.

### Table 15:

Compounds of the general formula I-A in which A is A2, R² is Cl, R³ is hydrogen and R¹ and B for each individual compound correspond in each case to one row of Table A.

### Table 16:

Compounds of the general formula I-A in which A is A2, R² is F, R³ is hydrogen and R¹ and B for each individual compound correspond in each case to one row of Table A.

### Table 17:

Compounds of the general formula I-A in which A is A2, R² is hydrogen, R³ is methyl and R¹ and B for each individual compound correspond in each case to one row of Table A.

### Table 18:

Compounds of the general formula I-A in which A is A2, R², R³ are methyl and R¹ and B for each individual compound correspond in each case to one row of Table A.

### Table 19:

Compounds of the general formula I-A in which A is A2, R² is Cl, R³ is methyl and R¹ and B for each individual compound correspond in each case to one row of Table A.

### Table 20:

Compounds of the general formula I-A in which A is A2, R² is F, R³ is methyl and R¹ and B for each individual compound correspond in each case to one row of Table A.

### Table 21:

Compounds of the general formula I-A in which A is A2, R² is hydrogen, R³ is ethyl and R¹ and B for each individual compound correspond in each case to one row of Table A.

### Table 22:

Compounds of the general formula I-A in which A is A2, R² is methyl, R³ is ethyl and R¹ and B for each individual compound correspond in each case to one row of Table A.

### Table 23:

Compounds of the general formula I-A in which A is A2, R² is Cl, R³ is ethyl and R¹ and B for each individual compound correspond in each case to one row of Table A.

### Table 24:

Compounds of the general formula I-A in which A is A2, R² is F, R³ is ethyl and R¹ and B for each individual compound correspond in each case to one row of Table A.

Very particular preference is given to the following thiazolecarboxanilides of the formula I:
N-(3′,4′,5′-trifluorobiphenyl-2-yl)-2-methyl-4-trifluoromethylthiazole-5-carboxamide,
N-(2′,4′, 5′-trifluorobiphenyl-2-yl)-2-methyl-4-trifluoromethylthiazole-5-carboxamide,
N-(3′,4′,5′-trifluorobiphenyl-2-yl)-2,4-dimethylthiazole-5-carboxamide and
N-(2′,4′, 5′-trifluorobiphenyl-2-yl)-2,4-dimethylthiazole-5-carboxamide.

The compounds I are suitable as fungicides. They are distinguished by an outstanding effectiveness against a broad spectrum of phytopathogenic fungi, especially from the class of the Ascomycetes, Deuteromycetes, Oomycetes and Basidiomycetes. Some are systemically effective and they can be used in plant protection as foliar fungicides, as fungicides for seed dressing and as soil fungicides.

They are particularly important in the control of a multitude of fungi on various cultivated plants, such as wheat, rye, barley, oats, rice, corn, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, such as, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, such as, for example, Cochliobolus sativus on cereals, Cochliobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumber plants;
Fusarium and Verticillium species on various plants, such as, for example, F. graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onion;
Phakopsara pachyrhizi and Phakopsara meibomiae on soybeans;
Phomopsis species on soybeans and sunflowers;
Phytophthora infestans on potatoes and tomatoes;
Phytophthora species on various plants, such as, for example, P. capsici on bell pepper;
Plasmopara viticola on grapevines;
Podosphaera leucotricha on apple;
Pseudocercosporella herpotrichoides on cereals;
Pseudoperonospora on various plants, such as, for example, P. cubensis on cucumber or P. humili on hops;
Puccinia species on various plants, such as, for example, P. triticina, P. striformins, P. hordei or P. graminis on cereals or P. asparagi on asparagus;
Pyricularia oryzae, Corticium sasakii, Sarocladium oryzae, S. attenuatum, Entyloma oryzae on rice;

Pyricularia grisea on lawns and cereals;

Pythium spp. on lawns, rice, corn, cotton, oilseed rape, sunflowers, sugar beet, vegetables and other plants, such as, for example, P. ultiumum on various plants, P. aphanidermatum on lawns;

Rhizoctonia species on cotton, rice, potatoes, lawns, corn, oilseed rape, potatoes, sugar beet, vegetables and on various plants, such as, for example, R. solani on beet and various plants;
Rhynchosporium secalis on barley, rye and triticale;
Scierotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, such as, for example, U. maydis on corn;
Venturia species (scab) on apples and pears, such as, for example, V. inaequalis on apple.

The compounds are particularly suitable for controlling harmful fungi from the class of the Peronosporomycetes (syn.Oomycetes), such as Peronospora species, Phytophthora species, Plasmopara viticola, Pseudoperonospora species and Pythium species.

The compounds I are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds or materials to be protected against fungal attack or the soil with a fungicidally effective amount of the active compounds. Application can be both before and after the infection of the materials, plants or seeds by the fungi.

The fungicidal compositions generally comprise between 0.1 and 95% by weight, preferably between 0.5 and 90% by weight, of active compound.

When employed in crop protection, the application rates are, depending on the kind of effect desired, between 0.01 and 2.0 kg of active compound per ha.

In seed treatment, the amounts of active compound required are generally from 1 to 1000 g/100 kg of seed, preferably from 5 to 100 g/100 kg of seed.

When used in the protection of materials or stored products, the active compound application rates depend on the kind of application area and on the desired effect. Amounts typically applied in the protection of materials are, for example, from 0.001 g to 2 kg, preferably from 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso® products, xylene), paraffins (for example mineral oil fractions), alcohols (for example methanol, butanol, pentanol, benzyl alcohol), ketones (for example cyclohexanone, gamma-butyrolactone), pyrrolidones (N-methylpyrrolidone, N-octylpyrrolidone), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example finely divided silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of a compound I according to the invention are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of a compound I according to the invention are dissolved in 70 parts by weight of cyclohexanone with addition of 10 parts by weight of a dispersant, for example polyvinylpyrrolidone. Dilution with water gives a dispersion. The active compound content is 20% by weight.

### C Emulsifiable Concentrates (EC)

15 parts by weight of a compound I according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of a compound I according to the invention are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of a compound I according to the invention are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of a compound I according to the invention are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of a compound I according to the invention are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations (GF)

20 parts by weight of a compound I according to the invention, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

### 2. Products to be Applied Undiluted

### J Dusts (DP, DS)

5 parts by weight of a compound I according to the invention are ground finely and mixed intimately with 95 parts by weight of finely divided kaolin. This gives a dustable product with an active compound content of 5% by weight.

### K Granules (GR, FG, GG, MG)

0.5 part by weight of a compound I according to the invention is ground finely and associated with 99.5 parts by weight of carriers. Current methods are extrusion, spray-drying or the fluidized bed. This gives granules with an active compound content of 0.5% by weight to be applied undiluted.
L ULV solutions (UL)
10 parts by weight of a compound I according to the invention are dissolved in 90 parts by weight of an organic solvent, for example xylene. This gives a product with an active compound content of 10% by weight to be applied undiluted.

Water-soluble concentrates (LS), suspensions (FS), dusts (DS), water-dispersible and water-soluble powders (WS, SS), emulsions (ES), emulsifiable concentrates (EC) and gel formulations (GF) are usually used for the treatment of seed. These formulations can be applied to the seed in undiluted or, preferably, diluted form. The application can be carried out before sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds I according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac® LF 300 and Lutensol® ON 30; EO-PO block polymers, for example Pluronic RPE® 2035 and Genapol® B; alcohol ethoxylates, for example Lutensol XP® 80; and sodium dioctylsulfosuccinate, for example Leophen® RA.

The compositions according to the invention in the application form as fungicides can also be present together with other active compounds, for example with herbicides, insecticides, growth regulators such as prohexadione Ca, fungicides or else with fertilizers. When mixing the compounds I or the compositions comprising them with one or more further active compounds, in particular fungicides, it is in many cases possible, for example, to widen the activity spectrum or to prevent the development of resistance. In many cases, synergistic effects are obtained.

The following list of fungicides with which the compounds according to the invention can be applied together is meant to illustrate the possible combinations, but not to limit them:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloro-isothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chloro-phenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propyl-chromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

### N-(3′,4′,5′-Trifluorobiphenyl-2-yl)-2-methyl-4-trifluoromethylthiazole-5-carboxamide (Ex. I.1)

At room temperature, 0.42 g of 3′,4′,5′-trifluorobiphenyl-2-ylamine and 0.72 g of bis(2-oxo-3-oxazolidinyl)phosphoryl chloride were added to a solution of 0.40 g of 2-methyl-4-trifluoromethylthiazole-5-carboxylic acid and 0.38 g of triethylamine in 30 ml of dichloromethane. The mixture was stirred at room temperature for 16 hours. It was then washed successively twice with dilute hydrochloric acid, twice with aqueous sodium bicarbonate solution and once with water. The organic phase was dried and concentrated. The crude product was purified by silica gel column chromatography using cyclohexane/methyl tert-butyl ether 1:2. This gave 0.61 g of the desired product in the form of light-brown crystals of m.p. 148-152° C.

The compounds of the general formula I, in which A is A1, listed in Table 25 below were prepared by the procedures given here.

TABLE 25

| | | | | | | | | Characterization |
|---|---|---|---|---|---|---|---|---|
| | | | | | | | | (m.p. or |
| Example | R¹ | R² | R³ | X | Y | p | w | ¹H-NMR) |
| I.1 | CF₃ | CH₃ | H | 3,4,5-F₃ | — | 0 | O | 148-152° C. |
| I.2 | CF₃ | CH₃ | H | 2,4,5-F₃ | — | 0 | O | 112-116° C. |
| I.3 | CH₃ | CH₃ | H | 3,4,5-F₃ | — | 0 | O | 123-128° C. |
| I.4 | CH₃ | CH₃ | H | 2,4,5-F₃ | — | 0 | O | 150-154° C. |

### Examples of the Action against Harmful Fungi

The fungicidal activity of the compounds of the formula I was demonstrated by the following tests:

The active compounds were prepared as a stock solution comprising 25 mg of active compound which was filled up to 10 ml with a mixture of acetone and/or dimethyl sulfoxide and the emulsifier Uniperol® EL (wetting agent having an emulsifying and dispersing action based on ethoxylated alkylphenols) in a solvent/emulsifier volume ratio of 99 to 1. The solution was then made up to 100 ml with water. This stock solution was diluted with the solvent/emulsifier/water mixture described to the active compound concentration given below.

### Use Example 1

### Curative Activity against Brown Rust of Wheat Caused by Puccinia recondita

Leaves of potted wheat seedlings of the cultivar “Kanzler” were inoculated with a spore suspension of brown rust (Puccinia recondita). The pots were then placed in a chamber with high atmospheric humidity (90 to 95%) and 20 to 22° C. for 24 hours. During this time, the spores germinated and the germ tubes penetrated into leaf tissue. The next day, the infected plants were sprayed to runoff point with the active compound solution described above at the active compound concentration stated below. After the spray coating had dried on, the test plants were cultivated in a greenhouse at temperatures between 20 and 22° C. and 65 to 70% relative atmospheric humidity for 7 days. The extent of the rust fungus development on the leaves was then determined.

In this test, the plants which had been treated with 250 mg/l of the compounds 1.1, 1.2, 1.3 and 1.4 from Table 25 showed an infection of at most 1%, whereas the untreated plants were 90% infected.

### Use Example 2

### Activity against Early Blight of Tomato Caused by Alternaria solani

Leaves of potted plants of the cultivar “Goldene Königin” were sprayed to runoff point with an aqueous suspension having the concentration of active compounds stated below. The next day, the leaves were infected with an aqueous spore suspension of Alternaria solani in 2% biomalt solution having a density of 0.12×10⁶ spores/ml. The plants were then placed in a water-vapor-saturated chamber at temperatures of between 20 and 22° C. After 5 days, the infection on the untreated, but infected control plants had developed to such an extent that the infection could be determined visually in %.

In this test, the plants which had been treated with 4 ppm of the compound I.1 showed an infection of at most 7%, whereas the untreated plants were 90% infected.

The plants which had been treated with 4 ppm of the comparative compound

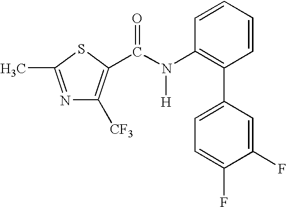

known from WO 2003/066609 showed an infection of 20%.

1. A thiazolecarboxanilide of the formula I 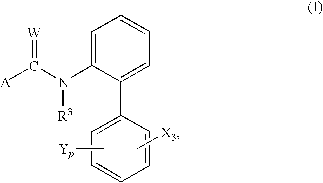
in which the variables are as defined below:
A is 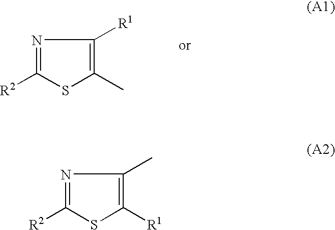
X is halogen;
Y is cyano, nitro, C₁-C₄-alkyl, C₁-C₄-haloalkyl, methoxy or methylthio;
p is 0 or 1;
R¹ is hydrogen, halogen, C₁-C₄-alkyl or C₁-C₄-haloalkyl;
R² is hydrogen, methyl or halogen;
R³ is hydrogen, methyl or ethyl;
W is oxygen or sulfur.

2. The thiazolecarboxanilide of the formula I according to claim 1 in which the variables are as defined below:
X is F or chlorine;
Y is C₁-C₄-alkyl, C₁-C₄-haloalkyl or methoxy;
p is 0, 1;
R¹ is hydrogen, halogen, C₁-C₄-alkyl or C₁-C₄-haloalkyl;
R² is hydrogen, methyl or halogen;
R³ is hydrogen or methyl;
W is oxygen.

3. The thiazolecarboxanilide of the formula I according to claim 1 in which the variables are as defined below:
X is F or chlorine;
Y is methyl, difluoromethyl, trifluoromethyl or methoxy;
p is 0 or 1;
R¹ is hydrogen, F, Cl, methyl, fluoromethyl, difluoromethyl, chlorofluoromethyl, chlorodifluoromethyl, dichlorofluoromethyl or trifluoromethyl;
R² is hydrogen, F, Cl or methyl;
R³ is hydrogen or methyl;
W is oxygen.

4. The thiazolecarboxanilide of the formula I according to claim 1 in which the variables are as defined below:
X is F or chlorine;
p is zero;
R¹ is hydrogen, F, Cl, methyl, fluoromethyl, difluoromethyl, chlorofluoromethyl, chlorodifluoromethyl, dichlorofluoromethyl or trifluoromethyl;
R² is hydrogen, F, Cl or methyl;
R³ is hydrogen;
W is oxygen.

5. The thiazolecarboxanilide of the formula I according to claim 1 in which the variables are as defined below:
X is F or chlorine;
p is zero;
R¹ is hydrogen, F, Cl, methyl, fluoromethyl, difluoromethyl, chlorofluoromethyl, chlorodifluoromethyl, dichlorofluoromethyl or trifluoromethyl;
R² is hydrogen, Cl or methyl;
R³ is hydrogen;
W is oxygen.

6. The thiazolecarboxanilide of the formula I according to claim 1 in which the substituent A has the meaning A1.

7. The thiazolecarboxanilide of the formula I according to claim 1, selected from the group consisting of N-(3′,4′,5′-trifluorobiphenyl-2-yl)-2-methyl-4-trifluoromethylthiazole-5-carboxamide, N-(2′,4′,5′-trifluorobiphenyl-2-yl)-2-methyl-4-trifluoromethylthiazole-5-carboxamide, N-(3′,4′,5′-trifluorobiphenyl-2-yl)-2,4-dimethylthiazole-5-carboxamide and N-(2′,4′,5′-trifluorobiphenyl-2-yl)-2,4-dimethylthiazole-5-carboxamide.

8. A composition for controlling harmful fungi, which comprises a fungicidal amount of at least one compound of the formula I according to claim 1 and at least one inert additive.

9. A method for controlling phytopathogenic harmful fungi which comprises treating the harmful fungi, their habitat and/or the materials, plants, the soil or seed to be protected against fungal attack with a fungicidally effective amount of at least one compound of the formula I according to claim 1.

10. The use of a compound I according to claim 1 for controlling phytopathogenic harmful fungi.

11. A seed, comprising at least one compound of the formula I according to claim 1 in an amount of from 1 to 1000 g/100 kg of seed.
